(19) United States
(12) Patent Application Publication
(10) Pub. No.: US 2022/0211331 A1
(43) Pub. Date: Jul. 7, 2022

(54) DETECTION OF INFECTION IN A PATIENT

(71) Applicant: Medtronic, Inc., Minneapolis, MN (US)

(72) Inventors: Holly S. Norman, Chisago City, MN (US); Douglas A. Hettrick, Andover, MN (US); Mark J. Phelps, Scottsdale, AZ (US); Shantanu Sarkar, Roseville, MN (US); Todd M. Zielinski, Ham Lake, MN (US)

(21) Appl. No.: 17/645,433

(22) Filed: Dec. 21, 2021

Related U.S. Application Data
(60) Provisional application No. 63/133,628, filed on Jan. 4, 2021.

(51) Int. Cl. A61B 5/00; A61B 5/053; A61B 5/01; A61B 5/11; A61B 5/08; A61B 5/024; A61B 5/021; A61B 5/145; G16H 50/30

(52) U.S. Cl. CPC A61B 5/7275; A61B 5/053; A61B 5/0066; A61B 5/01; A61B 5/1118; G16H 50/30; A61B 5/02405; A61B 5/021; A61B 5/14532; A61B 5/4839; A61B 5/0816

(57) ABSTRACT

This disclosure is directed to techniques for identifying a medical condition, such as an infection and/or a disease, from sensor data indicative of physiological parameters. In some examples, one example technique for identifying the medical condition includes process sensor data comprising data indicative of a plurality of physiological parameters for a patient comprising an impedance parameter, computing an index based upon values corresponding to at least two of the physiological parameters and based upon a comparison between the index and prediction criterion, generating, for display, output data corresponding to the comparison results, wherein the output data indicates a prediction of the medical condition in the patient if the comparison results indicate satisfaction of the prediction criterion.

This application claims the benefit of U.S. Provisional Application No. 63/133,628, filed Jan. 4, 2021, the entire content of which is incorporated herein by reference.

### FIELD

The disclosure relates generally to medical systems and, more particularly, medical device, technique, or system configured to detect medical conditions in patients.

### BACKGROUND

Complications related to infection, whether as a result of an immunocompromised state or an exposure to a virus, are clinically detrimental to patient health in general. Infections also negatively affect patients with one or more specific maladies (e.g., a patient with severe Chronic Obstructive Pulmonary Disease (COPD) and/or infected by a variant of the coronavirus (i.e., COVID).

Cancer, while fatal for some patients and treatable for other patients, compels both sets of patients to undergo harmful (and occasionally, life-threatening) therapies to avoid death and these therapies may exacerbate infection-related complications and the extent to which these complications negatively affect patient health. Chemotherapy—a therapy with possibly brutal side effects and social consequences that can reduce its efficacy—remains a mainstay of cancer therapy. Side effects of chemotherapy (e.g., sepsis, cardiotoxicity, and/or the like) can lead to long-term cardiac conditions, one of which is heart failure, and can affect patients years following cancer remission, requiring long-term follow-up monitoring. Due to the occurrence of cardiac-related complications post-cancer, the follow-up regimen for many cancer patients involves a yearly echocardiogram and overall assessment of cardiac health.

### SUMMARY

In general, the disclosure is directed to techniques for using a plurality of sensors to monitor patients for various conditions associated with cancer and treatment of cancer, e.g., development of infection, or conditions that result in a similar immunocompromised state. In some examples, the sensors are incorporated in a single implantable or wearable device. In some examples, the techniques facilitate remote monitoring of the patient. Example conditions include septic infections as well as cardiotoxicity and other conditions. Sepsis is a disease caused by an inflammatory response to an infection. While the following describes techniques for monitoring the patient for a specific infection, including sepsis, the techniques described herein may be applicable to monitoring and identifying other infections and diseases including precursors to septic infections (e.g., SIRS (systemic inflammatory distress syndrome) or ARDS (acute respiratory distress syndrome)) and precursors to other infections and diseases. Some techniques may be configured to monitor the patient and make predictions regarding the patient's general health. Any delay in identifying these infections and diseases may be fatal to the patient. A technique capable of providing real-time feedback on the patient's health may be used to quickly identify precursors to (e.g., septic) infections and diseases, thereby reducing the risks to the patient caused by having the actual infections and diseases. Real-time feedback may include any patient information recorded under hospital protocols (e.g., a number of hours from admission to administration of first IV antibiotic dose). Hence, implementing this technique provides an advantage to any medical system configured for patient monitoring and heath event detection.

With respect to septic infections, some techniques detect early signs of sepsis in individuals undergoing chemotherapy by monitoring levels of a plurality of physiological parameters of the individual. Some techniques accomplish remote patient monitoring by having one or more sensors (periodically) sensing physiological parameters. An example sensor may capture one or more signals from which at least some of parameter data is derived. Some techniques are directed to guiding the patient's sepsis treatment by determining an appropriate amount of therapy to administer the patient. Such guidance may pertain to sepsis treatment at any point during the patient's treatment (e.g., chemotherapy) while some techniques provide guidance regarding therapy to be applied before or after chemotherapy. Detecting and treating septic infections (including any precursor infections) in an accurate and timely manner (especially for cancer patients before, during, or after chemotherapy) prevents hypotension or septic shock, potentially avoiding hospitalization and/or death. The techniques of this disclosure may advantageously enable (e.g., post-cancer treatment) remote patient monitoring in general (or for specific conditions) and improved accuracy and efficiency in the detection and treatment of septic infections (e.g., administration of therapy) and, consequently, better evaluation of the condition of the patient.

In one example technique for monitoring a patient and identifying a medical condition, such as an infection and/or a disease, from sensor data indicative of the patient's physiological parameters, a method comprises processing sensor data comprising data indicative of a plurality of physiological parameters for a patient comprising an impedance parameter, computing an index for the medical condition based upon values corresponding to at least two of the physiological parameters and based upon a comparison between the index and prediction criterion, and generating, for display, output data corresponding to the comparison results, wherein the output data indicates a prediction of the medical condition in the patient if the comparison results indicate satisfaction of the prediction criterion.

In another example, a medical system comprises: one or more sensors configured to sense a plurality of physiological parameters for a patient; sensing circuitry coupled to the one or more sensors and configured to generate sensor data comprising data indicative of the plurality of physiological parameters comprising an impedance parameter corresponding to fluid accumulation; and processing circuitry configured to: compute an infection index (e.g., a sepsis index) based upon values corresponding to the impedance parameter and at least one other of the plurality of physiological parameters; and based upon a comparison between the infection index and infection prediction criterion (e.g., sepsis prediction criterion), generate, for display, output data corresponding to the comparison results, wherein the output data indicates a prediction of infection (e.g., sepsis) in the patient if the comparison results indicate satisfaction of the infection prediction criterion.

In another example, a non-transitory computer-readable storage medium comprising program instructions that, when executed by processing circuitry of a medical system, cause the processing circuitry to: process sensor data comprising data indicative of a plurality of physiological parameters for a patient comprising an impedance parameter; compute an infection index based upon values corresponding to an impedance parameter and at least one other of the plurality of physiological parameters; and based upon a comparison between the infection index and infection prediction criterion, generate, for display, output data corresponding to the comparison results, wherein the output data indicates a prediction of infection in the patient if the comparison results indicate satisfaction of the infection prediction criterion.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient.

FIG. 2 is a functional block diagram illustrating an example configuration of the implantable medical device (IMD) of the medical system of FIG. 1.

FIG. 3 is a conceptual side-view diagram illustrating an example configuration of the IMD of FIGS. 1 and 2.

FIG. 4 is a functional block diagram illustrating an example configuration of the external device of FIG. 1.

FIG. 5 is a block diagram illustrating an example system that includes an access point, a network, external computing devices, such as a server, and one or more other computing devices, which may be coupled to the IMD and external device of FIGS. 1-4.

FIG. 6A is a conceptual drawing illustrating a front view of a patient with another example medical system.

FIG. 6B is a conceptual drawing illustrating a side view of the patient with the example medical system of FIG. 6A.

FIG. 6C is a conceptual drawing illustrating a transverse view of the patient with the example medical system of FIG. 6A.

FIG. 7 is a flow diagram illustrating an example operation for determining whether a patient's physiological parameters indicates a septic infection.

FIG. 8 is a flow diagram illustrating an example operation for monitoring a patient's physiological parameters for medical conditions.

Like reference characters denote like elements throughout the description and figures.

### DETAILED DESCRIPTION

Medical systems as described herein encompass computerized hardware running software configured to perform various related tasks to patient health of which a number improve the patient's chances of overcoming (e.g., surviving) some medical condition. There are a number of example medical conditions being monitored and treated including a number of diseases and infections including those likely to cause harm. Medical systems such as those described herein are configured to protect patients from these diseases and infections and/or personalize treatments for at least some patients. Patients with compromised immune systems—patients having cancer and/or severe chronic conditions, such as advanced heart failure or advanced COPD (chronic obstructive pulmonary disease), organ transplant recipients, and/or the like—are even more likely to catch harmful diseases and infections and thus, have heightened standards of care. The medical systems described herein can be used to protect especially these patients by achieving and in some instances, exceeding those heightened standards of care.

To assess any given patient's health with respect to one or more medical conditions, medical systems may monitor the given patient's data indicative of one or more physiological parameters corresponding to patient activity (e.g., body movement), body temperature, respiration rate, tidal volume, neurological/physical pain data, heart rate, heart rate variability, arrhythmia burden, fluid accumulation, blood pressure or blood flow, tissue perfusion, and/or glucose level, as examples. Other physiological parameters may correspond to patient-reported symptoms, which may be submitted (e.g., uploaded) to the medical systems by an application running on a patient device. Combining at least some of these parameters may indicate the patient's likelihood of having a particular infection, disease, or other condition. The medical systems may analyze the data indicative of the one or more physiological parameters and based upon the patient's likelihood of having a particular infection or disease, determine whether that likelihood warrants some mediating action. The medical systems may perform various such actions including notifying the patient, a caregiver, or another entity (e.g., a remote monitoring service), for example, via various output (e.g., audio, text, and/or the like) and/or by communicating electronic messages to devices of the patient, the caregiver, or the other entity.

Values for any number of the above-mentioned physiological parameters may be derived from electronic signals, such as signals storing information associated with impedance, cardiac electrogram, acceleration, temperature, and/or optical coherence. Some example medical systems include one or more pairs of electrodes operative to monitor and capture example electrical signals and based on the captured signals, determine values for any of the above parameters. Some example medical systems leverage sensing equipment to capture example electrical signals (e.g., propagating signals or waves) from one or more types of sensors and based upon these captured signals, determines values for any number of physiological parameters. While some medical systems implement proprietary and/or third-party sensor(s) configured to sense the patient physiological parameters, some medical systems may incorporate various medical devices including (existing) implantable or wearable sensor technology to enable a number of additional measurements other than, for example, a cardiac EGM, which may be monitored by above pair(s) of electrodes.

To monitor and possibly detect different medical conditions, some medical systems compare different combinations of these values to various prediction criteria and in view of that comparison, determine whether those values satisfy the various prediction criteria. The satisfaction of any prediction criterion indicates a sufficient likelihood that the patient has the corresponding medical condition. A disease or infection prediction criterion, examples of the above prediction criterion, may include a criterion for predicting a septic infection and/or a precursor to sepsis. To illustrate, an example medical system may compute a single index or score as a mathematical combination of at least two parameters, compare that index with one or more thresholds corresponding to some medical condition (e.g., a disease or infection as described herein), and if that index exceeds the one or more thresholds, predict that medical condition for the patient's diagnosis. As a response, some medical systems may display for output indicating, as the patient's likely diagnosis, a detection of the predicted medical condition. The present disclosure describes, as one example, sepsis prediction criteria but at least one of the devices, techniques, or systems described herein may apply prediction criteria for other medical conditions.

Some of the medical systems described herein operate a monitoring algorithm that, in accordance with a schedule, performs various scheduled operations including periodic capturing of these electronic signals and/or a subsequent comparison of prediction criterion with values derived from the captured signals. As another scheduled operation, some medical systems may regularly update computations of the patient's likelihood of having the particular infection or disease based upon recent parameter values for the patient's physiological profile. In some examples, if, at any time, the patient's physiological profile satisfies the prediction criterion of the particular infection or disease (e.g., sepsis), the medical systems may engage in a number of alert and/or therapy protocols. One example medical system may notify, via communicated messages, some entity (e.g., the patient, the patient nurse, a hospital system) of the particular infection or disease and that notification may prompt the entity to deliver therapy to the patient. Another example medical system may notify, via communicated messages, an appropriate entity of the patient's adverse physiologic response. Other example medical systems may be configured to perform therapeutic actions. For example, a medical system may be adapted with various equipment configured to deliver therapy to the patient, for example, by administering dosage(s) of some treatment (e.g., antibiotics) or modulating an intensity of chemotherapy to mitigate the adverse physiologic response.

As described herein, a variety of types of medical devices sense cardiac EGMs; some of these medical devices are non-invasive, e.g., using a plurality of electrodes placed in contact with external portions of the patient, such as at various locations on the skin of the patient. The electrodes used to monitor the cardiac EGM in these non-invasive devices may be attached to the patient using an adhesive, strap, belt, or vest, as examples, and electrically coupled to a monitoring device, such as an electrocardiograph, Holter monitor, or another electronic device. The electrodes are configured to sense electrical signals associated with the electrical activity of the heart or other cardiac tissue of the patient, and to provide these sensed electrical signals to the electronic device for further processing and/or display of the electrical signals.

The non-invasive devices—in combination with the monitoring algorithm and other methods—may be utilized on a temporary basis, for example to monitor a patient during a clinical visit, such as during a doctor's appointment, or for example for a predetermined period of time, for example for one day (twenty-four hours), or for a period of several days. If an invasive device is to be used for the patient, that device may execute the monitoring algorithm at a different frequency than any non-invasive device. In other examples, the non-invasive devices may execute the monitoring algorithm at different rates. An example invasive device may apply the monitoring algorithm a higher rate, for example, during treatment or during episodes of elevated risk, when the patient is not feeling well, and/or as directed by the patient, the caregiver, and/or the clinician (as needed).

External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, available from Medtronic plc, of Dublin, Ireland. Such external devices may facilitate relatively longer-term monitoring of patients during normal daily activities, and may periodically transmit collected data to a network service, such as the Medtronic Carelink™ Network.

Implantable medical devices (IMDs) also sense and monitor cardiac EGMs. The electrodes used by IMDs to sense cardiac EGMs are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic plc. Some IMDs that do not provide therapy, e.g., implantable patient monitors, sense cardiac EGMs. One example of such an IMD is the Reveal LINQ™ Insertable Cardiac Monitor, available from Medtronic plc, which may be inserted subcutaneously. Such IMDs may facilitate relatively longer-term monitoring of patients during normal daily activities, and may periodically transmit collected data to a network service, such as the Medtronic Carelink™ Network.

In implantable or wearable sensor technologies such as those described above, a biosensor implanted in the patient (e.g., an insertable cardiac monitor like LINQ™) may measure impedance (e.g., of electrodes), electrograms (e.g., differential voltage), acceleration (e.g., 3-dimensional), temperature, and/or optical coherence (e.g., correlation between propagating signals or waves) and these measurements provide patient physiological parameter data. Wireless telemetry of the measurements from the biosensor enables the patient's data to be compiled, stored, and analyzed remotely over seconds, minutes, days, months, and years. These additional measurements may enable new practical applications for existing implantable or wearable sensor technologies. Early indicators of a number of medical conditions include a change in one or more physiological parameters, a rate of change of one or more physiological parameters, and/or an amount of change in one or more physiological parameters. Changes may occur over hours or over days (e.g., where fluctuations within one day are normal, but a change in daily fluctuations is not).

Based upon at least some of the patient physiological parameter data provided by implantable or wearable sensor technologies, medical systems such as those described herein may monitor immune-compromised patients for declines in health, for example, before/during/after treatment for a major illness such as a type of cancer. Regardless of which type or types of devices are used, patients receiving post-cancer treatment(s) (e.g., chemotherapy) are at great risk of developing an infectious disease and, possibly, succumbing to that disease. For at least this reason, the medical systems described herein may be employed for monitoring the patient's physiological parameter(s) and/or detecting possible diseases and infections in that patient. Monitoring may occur over several stages of chemotherapy, such as before, during, and/or after chemotherapy. Monitoring may also detect other negative health consequences such as arrhythmias, sepsis, cardiotoxicity, and other infections. Monitoring may even detect the patient's lung infection, for example, based on an altered respiration rate, effort, and pattern and/or fluid accumulation in the patient's lung.

In this manner, the techniques of this disclosure may advantageously leverage sensing technologies for monitoring a patient to identify a variety of medical conditions associated with cancer and treatment of cancer.

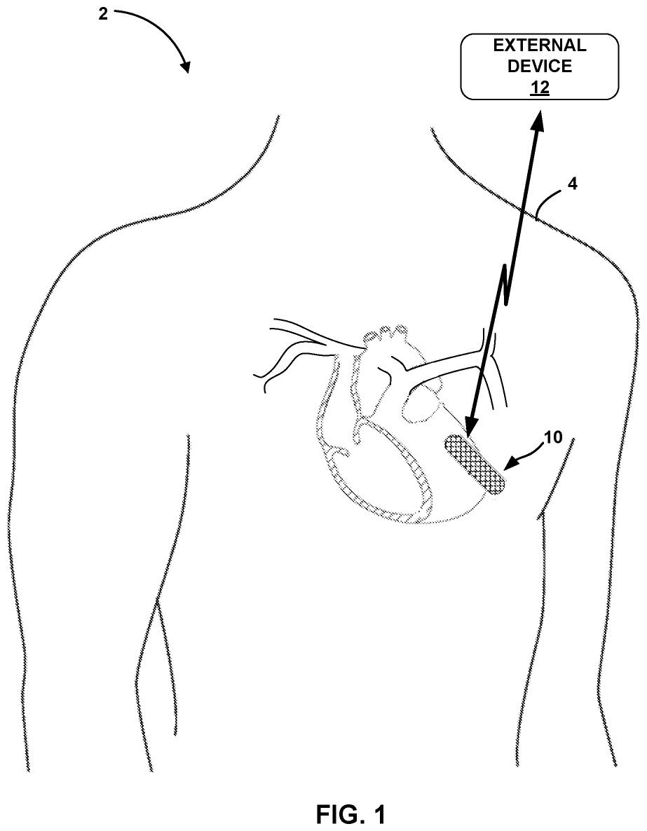

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1), and is configured to sense a cardiac EGM via the plurality of electrodes. In some examples, IMD 10 takes the form of the LINQ™ ICM available from Medtronic, Inc. of Minneapolis, Minn. IMD 10 includes one or more sensors configured to sense patient activity, e.g., one or more accelerometers.

External device 12 may be a computing device with a display viewable by the user and an interface for receiving user input to external device 12. In some examples, external device 12 may be a notebook computer, tablet computer, workstation, one or more servers, cellular phone, personal digital assistant, or another computing device that may run an application that enables the computing device to interact with IMD 10.

External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., radiofrequency (RF) telemetry according to the 802.11 or Bluetooth® specification sets, or other communication technologies operable at ranges greater than near-field communication technologies).

External device 12 may be used to configure operational parameters for IMD 10. External device 12 may be used to retrieve data from 1 MB 10. The retrieved data may include values of physiological parameters measured by IMD 10, physiological signals recorded by 1 MB 10, indications of medical conditions (e.g., sepsis) detected by IMD 10 among other information types. Other than sepsis (including septic infections), the retrieved data may include indications of detectable medical conditions covering a number of infections and diseases including any infection and/or disease known for afflicting sensitive and/or susceptible people such as those with compromised immune systems. The retrieved data may include indications of, for example, cardiotoxicity, which is prevalent amongst cancer patients including those in treatment and in recovery.

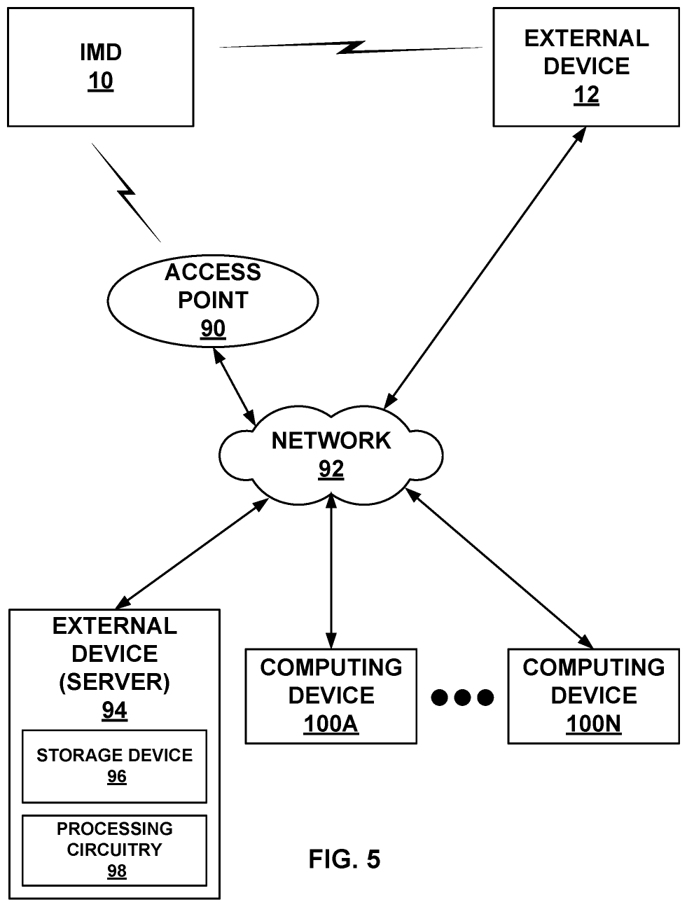

The retrieved data may include indications of episodes of arrhythmia, asystole, or other maladies including harmful diseases and infections. For example, external device 12 may retrieve cardiac EGM segments recorded by IMD 10 due to IMD 10 determining that an episode of asystole or another malady occurred during the segment. While IMD 10 may determine the episode of asystole from the cardiac EGM segments, IMD 10 may have access to sensor data other than cardiac EGM segments and may use that sensor data to gauge whether the episode of asystole was determined correctly. On the other hand, external device 12 may receive signals from one or more types of sensors and generate sensor data for use in determining whether patient most likely has a specific medical condition, such as the episode of asystole, a septic infection, and/or the like. For example, external device 12 may retrieve any metric value, including measurements related to the medical condition determination, in addition to determination analysis data in accordance with the techniques described herein from IMD 10. As will be discussed in greater detail below with respect to FIG. 5, one or more remote computing devices may interact with 1 MB 10 in a manner similar to external device 12, e.g., to program IMD 10 and/or retrieve data from 1 MB 10, via a network.

Processing circuitry of medical system 2, e.g., of IMD 10, external device 12, and/or of one or more other computing devices, may be configured to perform the example techniques for detecting changes in patient health of this disclosure. In some examples, medical conditions may cause patient health to decline, for example, due to a disease and/or infection. Processing circuitry of medical system 2 may be communicably coupled to one or more sensors, which may be devices each configured to sense patient physiological parameters in some form, and sensing circuitry configured to generate sensor data. In some examples, the processing circuitry of medical system 2 analyzes the sensor data generated by the sensing circuitry and associated with physiological parameter(s) to determine whether one or more of a plurality of prediction criterion are satisfied. The plurality of prediction criterion may include at least one criterion for each potential medical condition. In some instances, processing circuitry of medical system 2 may utilize the prediction criterion to access analyzed sensor data and then, possibly make an initial prediction of a certain medical condition and/or gauge an accuracy of an initial predicted medical condition. Each of the prediction criterion may be configured to detect one or more indicators of prevalent infections and/or diseases.

In some examples of medical system 2, an implanted biosensor may measure impedance, electrograms or electrocardiograms, acceleration, temperature, and/or optical coherence. The implanted biosensor may be components of IMD 10 or may be a separate device. Sensing circuitry of medical system 2 may captures these measurements as sensor data and among these measurements, identifies values for physiological parameters. Processing circuitry of medical system 2, e.g., of IMD 10, external device 12, and/or of one or more other computing devices, may be configured to monitor and collect samples of the physiological parameters over a period of time.

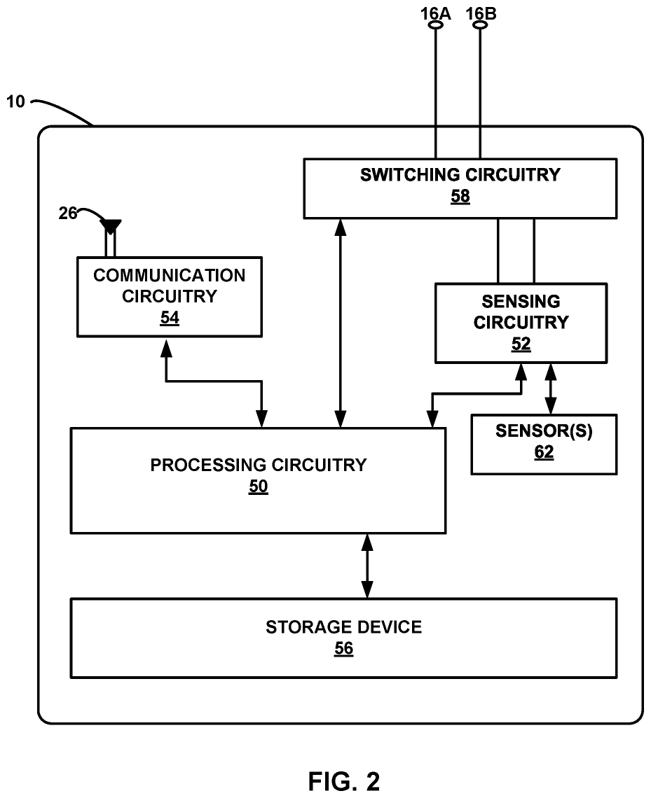

In general, optical coherence refers to a statistical similarity of an optical wave field at two points in space or time and, specifically, describes various correlation properties between physical quantities of example signals or waves. A cross-correlation function may be used to quantify the coherence of two waves; for example, the cross-correlation function may determine how well correlated the waves are, for instance, in terms of coherence length (e.g., spatial coherence) and/or coherence time (temporal coherence). The biosensor may determine that a pair of propagating signals or waves are perfectly coherent if both have identical frequencies and/or waveforms and a constant phase difference, but if the phase difference is not constant, the pair of propagating signals or waves may only be partially coherent. To illustrate by way of example, the biosensor (e.g., in operation of an optic instrument (e.g., an interferometers) or a similar sensor such as a wavefront sensor) may measure the optical coherence of an incoming light field (e.g., light waves) from a portion of the patient's body (e.g., by way of reflection). This signal (e.g., an optical coherence signal) may be directly correlated to the level of blood oxygenation as in standard pulse oximetry (e.g., as depicted in FIG. 1). Changes in blood oxygenation could indicate a change in the patient's overall treatment status and his/her response to a current or recent treatment. Likewise, a wave morphometry of the optical coherence signal varies with the patient's cardiac cycle and is generally similar to arterial blood pressure (e.g., as depicted in FIG. 2). Therefore, various features of the wave morphometry (e.g., a rate of decay in the diastolic period, a ratio of systolic to diastolic pulse amplitude, and/or the like) may change and at least some of those change may imply a change in the patient's overall treatment status or his/her response to the current or recent treatment. The biosensor may combine the optical coherence measurement(s) with impedance measurements to determine an accuracy of other measurements.

In some examples, electrogram or electrocardiogram (ECG) specific parameters may include a continuous QT interval, a PVC burden, QRST morphology changes (e.g., in terms of R-wave amplitude, width, ST segment, T-wave morphology and/or the like), chemical sensors for potassium or Creatinine, and/or the like. Q, R, S, and T are abbreviations for Q-waves, R-wave, S-wave, and T-waves.

Processing circuitry of medical system 2, e.g., of IMD 10, external device 12, and/or of one or more other computing devices, may be configured to compute a score or index using the values from the collected samples of the physiological parameters, compare that score with at least one prediction criterion for a specific medical condition and based upon that comparison, render a prediction of the specific medical condition in the patient if the comparison results indicate satisfaction of the prediction criterion. Some examples may implement a multivariate time series analysis algorithm. If the index or score meets a threshold, a notification is sent to the patient, to a member of the patient's community, or to a remote monitoring service associated with the patient. For example, the notification may be an educational message to the patient, an alarm to the patient's clinician, or a signal to a dispensing device to change a dosage of medicine for the patient.

Although described in the context of examples in which IMD 10 that senses the cardiac EGM comprises an insertable cardiac monitor, example systems including one or more implantable, wearable, or external devices of any type configured to sense a cardiac EGM may be configured to implement the techniques of this disclosure. In some examples, processing circuitry in a wearable device may execute same or similar logic as the logic executed by processing circuitry of IMD 10 and/or other processing circuitry as described herein. In this manner, a wearable device or other device may perform some or all of the techniques described herein in the same manner described herein with respect to IMD 10. For example, the wearable device may compute values (e.g., metric values) for the physiological parameters and then, analyze those values for indicia of certain medical conditions. Similar to processing circuitry of IMD 10, processing circuitry of the wearable device may analyze the sensor data to determine which parameter values to use in computing a score or index as data indicative of a likelihood of the patient having the medical condition. In some examples, the wearable device operates with IMD 10 and/or external device 12 as potential providers of computing/storage resources and sensors for monitoring patient activity in general and one or more patient physiological parameters in particular. For example, the wearable device may communicate the sensor data to external device 12 for storage in non-volatile memory and for determining value(s) for one or more patient physiological parameters.

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to sense electrical signals of the heart of patient 4, for example by selecting the electrodes 16 and polarity, referred to as the sensing vector, used to sense a cardiac EGM, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more accelerometers (e.g., a three-axis accelerometer), pressure sensors, a gyroscope, a temperature gauge, a moment transducer, and/or optical sensors, as examples. In some examples, sensing circuitry 52 may include one or more filters and amplifiers for filtering and amplifying signals received from electrodes 16 and/or sensors 62.

Sensing circuitry 52 may generate sensor data from signals received from sensor(s) 62 and that sensor data may indicate various patient physiological parameters. Sensing circuitry 52 and processing circuitry 50 may store the sensor data in storage device 56. Processing circuitry 50, executing logic configured to perform an algorithm (e.g., a detection or monitoring algorithm) on the sensor data, is operative to monitor for and detect any change (e.g., a decline) in patient health. Processing circuitry 50 may control one or more of sensors 62 to sense physiological parameters in some form. There are a number of methods for converting the sensor data into parameter data, which may be a value representing a quality or a quantity. Example physiological parameter values include a body temperature, a respiration rate, a tidal volume, a heart rate, a heart rate variability, an arrhythmia burden value, a fluid accumulation value, an activity (e.g., steps), a blood pressure, a glucose level, and/or the like.

Sensing circuitry 52 and/or processing circuitry 50 may be configured to detect cardiac depolarizations (e.g., P-waves or R-waves) when the cardiac EGM amplitude crosses a sensing threshold. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining heart rate and detecting arrhythmias, such as tachyarrhythmias and asystole.

In accordance with native functionality, processing circuitry 50 may detect an asystole episode based upon asystole detection criterion such as an absence of a cardiac depolarization for a threshold period of time. Processing circuitry 50 proceed to apply appropriate prediction criterion to the sensor data and validate the initial asystole episode detection. Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic Carelink™ Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include sensor data for suspected diseases, infections, and/or other medical conditions and/or indications of declines in health including indications of satisfaction of any one of various medical condition prediction criterion.

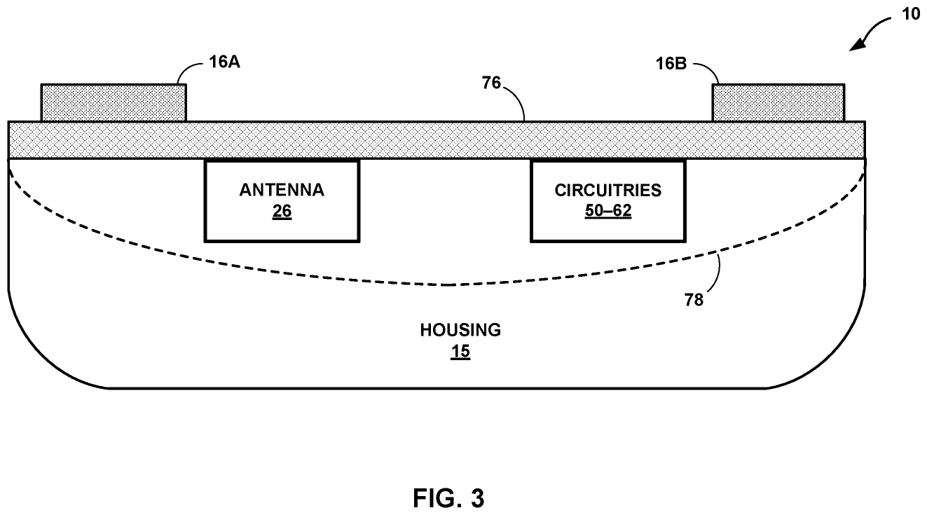

FIG. 3 is a conceptual side-view diagram illustrating an example configuration of IMD 10 of FIGS. 1 and 2. While different examples of IMD 10 may include leads, in the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. Certain ones of sensors 62 may be formed or placed on outer surface of cover 76 or on outside of housing 15. In the illustrated example, antenna 26 is formed or placed on the inner surface of cover 76, but may be formed or placed on the outer surface in some examples. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

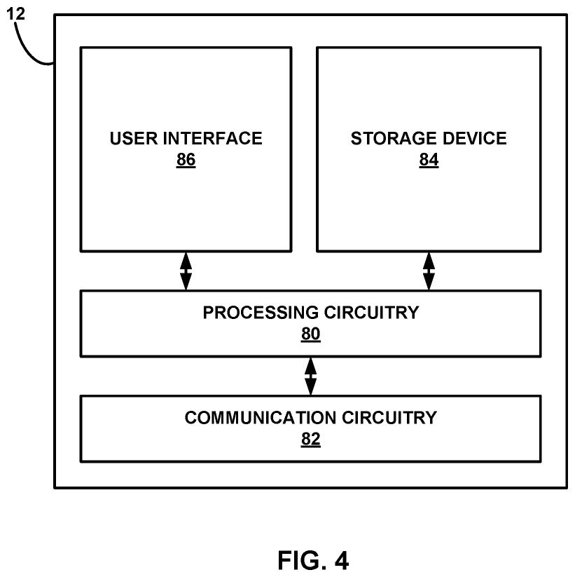

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, NFC, RF communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data to external device 12. In turn, external device 12 may receive the collected data from IMD 10 and store the collected data in storage device 84. The data external device 12 receives from IMD 10 may include sensor data including data corresponding to a plurality of physiological parameters for patient 4.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., representations of sensor data inclusive of data indicative of physiological parameters, indications of medical condition (predictions) based on determinations that one or more prediction criterion are satisfied by the data indicative of physiological parameters, indications of changes in the sensor data indicative of the physiological parameters, and indications of changes in patient health that correlated to the changes in the data indicative of the physiological parameters and/or the satisfaction of the one or more prediction criterion for each medical condition. Other examples of related information for presentation via user interface 86 may include computations of scores or indexes based upon at least a portion of the data indicative of the physiological parameters. Yet another example of related information for presentation via user interface 86 may be estimations of probability data for detectable medical conditions. Processing circuitry 80 may output, for display, the above-mentioned related information in a number of presentation formats such that user interface 86, as envisioned by the present disclosure, may be compatible with any device hardware, operating system, application platform, and/or the like. Example prediction criterion may include thresholds for at least some individual parameters and/or for combinations of the at least some individual parameters such that satisfying these thresholds may predict a corresponding medical condition; for example, if values corresponding to the at least some individual parameters or the combinations of the at least some individual parameters meets or exceeds the thresholds, processing circuitry 80 may output, for display, a positive prediction for the corresponding medical condition. In addition, user interface 86 may include an input mechanism configured to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

As described herein, external device 12 and IMD 10 may monitor a patient (e.g., a cancer patient) for conditions, such as possible infections or diseases. To illustrate by way of an example patient monitoring technique for detecting septic infections, processing circuitry 80 of external device 12 may execute the following operations to determine whether a patient's physiological parameters at least imply a septic infection for the patient and to determine whether that patient's physiological parameters require a mediating action. First, two or more types of sensor devices may generate multiple signals while sensing at least two physiological parameters (e.g., parameter levels, values, and other data). These sensor devices may include sensors 62 in IMD 10 and/or other sensors (e.g., external sensing equipment). Some examples of IMD 10 comprises one or more (implanted) biosensors (e.g., an insertable cardiac monitor such as LINQ from Medtronic) housing the two or more types of sensor devices, to sense any patient's physiological parameters by measuring impedance, electrograms, acceleration, temperature, and/or optical coherence. Wireless telemetry of the measurements from the biosensor enables the patient's parameter data to be compiled, stored over time, and analyzed remotely by external device 12.

Second, after sampling those signals over time, external device 12 and/or IMD 10 may generate sufficient parameter data for the septic infection detection (e.g., via a comparison with one or more criterion for predicting a positive septic infection). Early signs (e.g., a precursor) of septic infection include a change in one or more physiological parameters, a rate of change of one or more physiological parameters, or an amount of change in one or more physiological parameters. The physiological parameters include body temperature, respiration rate, tidal volume, heart rate, heart rate variability, arrhythmia burden, fluid accumulation (e.g., edema), activity (e.g., steps), blood pressure, glucose level, etc. The above-mentioned biosensor may provide measurements from which some of the at least one physiological parameters are derived. For example, a temperature sensor may provide temperature measurements for determining a value, which may be referred to as temperature parameter. Derived from sensed impedance measurements, fluid accumulation may be one example of an impedance parameter. Changes may occur over hours or over days (e.g., where fluctuations within one day are normal, but a change in daily fluctuations is not). In some examples, external device 12 and/or IMD 10 may store a significant amount of sample data in storage device 84 and/or storage device 56, respectively, to ensure accuracy in the parameters and confidence in any rendered prediction of a medical condition. The patient's temperature levels, for instance, may fluctuate over time but should converge to a same quantity over numerous samples.

Third, external device 12 may retrieve the parameter data from IMD 10 and processing circuitry 80 may analyze the parameter data to determine a likelihood that the septic infection currently afflicts the patient. In some examples, processing circuitry 80 of external device 12 leverages one or more septic infection prediction criterion including one or more specific sepsis-related parameter thresholds and/or a sepsis index or score threshold. Processing circuitry 80 of external device 12 may compute, using any suitable method, the sepsis index or score for the one or more septic infection prediction criterion; as an example, processing circuitry 80 of external device 12 may apply the sampled physiological parameter data to a multivariate time series analysis algorithm (e.g., a weighted average) to determine the sepsis index or score. Detecting a precursor to sepsis (e.g., SIRS, ARDS (acute respiratory distress syndrome), and/or the like), may encompass between 2-6 physiological parameters, such as temperature, heart rate, respiration rate, and activity sensors. Processing circuitry 80 of external device 12 may compare 2-6 parameters to individual parameter thresholds or incorporate the 2-6 parameters into an integrated algorithm to compute a score or index of SIRS detection. In some instances, only 2 of the 6 parameters must be elevated to transition a patient from SIRS to sepsis. Sepsis may be detected if the following one or more prediction criterion are met for at least X out of Y hours in a 24 hour period (e.g. X=3, y=12): Body Temperature >than 38° C. (100.4° F.) OR<36° C. (96.8° F.), AND Mean Heart Rate >90 bpm AND Respiratory rate >20 breaths per minute. Instead of absolute thresholds, the one or more prediction criterion may reflect relative changes of individual parameters, e.g. is the patient's heart rate or respiration rate increasing. In one example, processing circuitry 80 may accumulate differences between long term and short term averages to detect a relative change in a specific parameter.

Processing circuitry 80 of external device 12 may compute an infection index (such as the sepsis index or score mentioned above or an index or score corresponding to a sepsis precursor) to represent a mathematical combination of values corresponding to two or more parameters of a same sample period, compare that index or score with an infection prediction criterion (such as the sepsis index threshold criterion) and then, determine, based upon that comparison, whether to register a prediction of an infection. The sepsis index or score threshold criterion may be a pre-determined threshold value or may be determined based on other samples of patient parameter data. Processing circuitry 80 of external device 12 may compute a sepsis index or score for each sample period such that a first sepsis index or score is computed for a first time period, a second sepsis index or score is computed for a second time period, and so forth. Processing circuitry 80 of external device 12 may compute a difference value between consecutive sepsis indices (e.g., between the first sepsis index and the second sepsis index) and based upon a determination that the computed difference satisfies the sepsis threshold criterion, generate for display the output indicating the sepsis prediction for the patient. Processing circuitry 80 of external device 12 may compute a rate of change in sepsis indices over time and based upon a determination that the computed rate of change satisfies a sepsis threshold, generate for display the output indicating the sepsis prediction for the patient.

If the comparison results in the sepsis index/score exceeding the threshold criterion, processing circuitry 80 of external device 12 may determine whether the sepsis index/score satisfies the corresponding threshold criterion (e.g., based on whether a difference value is accurate and substantial for a positive septic infection prediction). If so, processing circuitry 80 of external device 12 may output, for display, data indicating a likely detection of the septic infection. If the sepsis index or score threshold criterion operates as a target (rather than a minimum) for the computed sepsis index/score to meet (rather than exceed), processing circuitry 80 of external device 12 may determine whether the computed sepsis index/score satisfies the threshold (e.g., whether the difference value is accurate and insubstantial for a positive septic infection prediction).

In some examples, processing circuitry 80 of external device 12 may build and train a machine learning model (e.g., a deep learning neural network) to predict a patient's likelihood of having a sepsis or any other disease or infection. Instead of comparing the patient's physiological parameter data to sepsis prediction criteria, the machine learning model may include a multi-variate function configured to compute the sepsis index/score. The multi-variate function may define a variable for each parameter and a weight for each parameter value.

As an alternative, processing circuitry 80 of external device 12 may build and train the machine learning model with features in addition to or instead of the patient's physiological parameters. Processing circuitry 80 of external device 12 may perform feature engineering to look for features in continuously measured signals (e.g., from sensors 62) and based on whether these features are above or below a threshold (or whether there is an increasing or decreasing trend), the machine learning model may provide a positive prediction or a negative prediction. Processing circuitry 80 of external device 12 may implement one or more techniques for mathematically combining the features into an index/score for predicting an infection or disease; some examples of these techniques include an X of Y scheme (e.g., where Y is the total number of criteria/parameters and X is the number of parameters that are out of range (or met certain criteria)), a data fusion technique such as linear or logistic regression, or a non-linear technique such as random forests or Bayesian Belief Networks.

In some examples, processing circuitry 80 of external device 12 may integrate a patient's physiological parameter data with information retrieved from EMR systems, such as a clinical history, lab results, diagnostic tests, medications, a recent clinical encounter history, and/or the like. Processing circuitry 80 of external device 12 may build and train a machine learning model using the integrated data. Instead of or in addition to the above-mentioned features or the patient's physiological parameters, processing circuitry 80 of external device 12 may build and train a machine learning model using only the data retrieved from the EMR systems.

As an option, processing circuitry 80 of external device 12 may also push data to the EMR systems. In addition to various patient data, processing circuitry 80 of external device 12 may enable the clinician to prescribe the patient appropriate a new treatment or modify a current treatment (and possibly, add a treatment schedule for the caregiver to administer the new/modified treatment). For an arrhythmia patient who has a bacterial infection, processing circuitry 80 of external device 12 may communicate a message to the EMR systems prescribing antibiotics to treat the patient's bacterial infection. In this manner, the clinician may view information for a prescription of antibiotics and compare that prescription with any antiarrhythmic agents or other medications prescribed to the patient to treat their symptoms of arrhythmias include heart palpitations, fainting, chest pain, and shortness of breath. To administer the anti-biotics, processing circuitry 80 of external device 12 may upload data for a method configured to guide the patient or caregiver regarding dosage time(s) and/or amount(s) through any given day. In general, processing circuitry 80 of external device 12 may push to the EMR systems data outlining steps of a customized method configured to instruct the patient on a next step.

Fourth, if the sepsis index or score satisfies (e.g., meets or exceeds) the threshold criterion, processing circuitry 80 of external device 12 may communicate a message to the patient (e.g., to a patient device including consumer devices), to a device for display to person given authority over the patient's care (e.g., a member of the patient's community/family, a caregiver, a doctor, or hospital staff), or to a medical device associated with the patient including IMD 10 or another medical device. Processing circuitry 80 of external device 12 may communicate the message (e.g., notification) to prompt the caregiver or the doctor to examine the patient to confirm and possibly treat the patient's septic infection. The notification may be an educational message to the patient, an alarm to the patient's clinician, or a signal to a dispensing device to change a therapy (e.g., a dosage of medicine or another treatment) for the patient. In a hospital setting, processing circuitry 80 of external device 12 may communicate the notification to the therapy dispensing device (e.g., a drug pump for IV antibiotics) that the patient is connected to and automatically alter medications/dosages in a closed loop fashion. Processing circuitry 80 of external device 12 may communicate the message to include the notification and a set of instructions for the patient and/or the person(s) with authority over the patient's care to follow. In some examples, the set of instructions may prescribe a treatment regimen including a treatment type, amount, and delivery procedure. In other examples, processing circuitry 80 of external device 12 may communicate the set of instructions in a control directive (e.g., an interface command, function call, or a setup parameter) to the medical device. To illustrate by way of example, if the patient also is a diabetic, the patient may have IMD 10 and an insulin pump to supply and delivery diabetes therapy. If the septic infection (negatively) affects the patient's diabetes (e.g., by disrupting the patient's blood sugar level and/or putting the patient into a state of diabetic shock that requires another person's help), processing circuitry 80 of external device 12 may communicate a message to notify the patient of the medical condition and possible disruption to diabetes therapy.

While FIG. 1 may depict examples of external device 12 that are communicably coupled to IMD 10, FIG. 4 depicts external device 12 where some examples are either not coupled to IMD 10 or are coupled to another cardiac monitor or another device altogether. For example, external device 12 may be configured to (remotely) monitor cancer recovery patients and provide them with a number of benefits. In general, processing circuitry 80 of external device 12 may apply various prediction criteria for numerous medical conditions that can be detected from the patient's physiological parameters. Early detection offers any patient time to access proper care. Close monitoring may be essential for an early detection and treatment of cardiovascular disease induced by antineoplastic treatment, and the various prediction criteria helps minimize serious acute and chronic cardiac consequences. For immune-compromised cancer patients, the various prediction criteria protect these patients and maintain their health for the rigors of chemotherapy.

Chemotherapy remains a mainstay of cancer therapy, but the therapy can have brutal side effects and social consequences that can reduce its efficacy. For example, one well-established side effect of chemotherapy is cardiotoxicity which can lead to short-term and long-term cardiac conditions, one of which is heart failure and can affect patients years following cancer remission, requiring long-term follow-up monitoring. One example serious (e.g., long-term) cardiac complication (e.g., side-effect) of anticancer therapy is CHF, with clinical presentation similar to CHF of other etiologies. While some specific classifications of chemotherapy are proarrhythmic, arrhythmias and/or atrial fibrillations may be short-term complications. The standard method of cardiac monitoring is LVEF assessment by echocardiography, MUGA (Multi Gated Acquisition) scan or cardiac MRI. Arrhythmias and conduction disorders involve mostly asymptomatic sinus bradycardia. Atrial fibrillation may be associated with the use of various cytotoxic agents. Putative patho-mechanism involves systemic inflammation related to cancer. In addition to septic infection detection as describe herein, processing circuitry 80 of external device 12 may apply, to the patient's physiological parameter data, one or more cardiotoxicity prediction criterion, which in accordance with an operational definition of cardiotoxicity, are as follows: (1) Cardiomyopathy characterized by a decrease in LVEF that was either global or more severe in the septum; (2) Symptoms of CHF; (3) Associated signs of CHF, including S3 gallop, tachycardia, or both; (4) Decline in LVEF of at least 5% to less than 55% with signs or symptoms of CHF, or a decline in LVEF of at least 10% to below 55% without signs or symptoms; and (5) Heart Rate irregularities. Such an operational definition could be easily adapted to implantable or wearable sensors, such as IMD 10, to monitor for chemotherapy cardiotoxicity.

In cooperation, IMD 10 and external device 12 may provide a remote monitoring solution that can reduce morbidity and prevent unplanned hospitalizations. To the patient's benefit, an example healthcare monitoring service (e.g., on behalf of the patient's health care provider) may enable monitoring across the healthcare service continuum, allowing multiple clinicians to manage the delivery of complicated medical procedures. In addition, many chemotherapy pharmaceuticals are pro-arrhythmic and juvenile cancer patients and their parents have increased anxiety levels while undergoing chemotherapy treatment. The experience with anthracycline cardiotoxicity proved that the early detection and treatment of cardiotoxicity could significantly reduce the development of clinical manifestations. Due to the occurrence of cardiac-related complications post-cancer treatment, external device 12 may suffice as the follow-up regimen for many cancer patients, e.g., instead of a yearly echocardiogram and overall assessment of cardiac health.

FIG. 5 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as indications of predictions of one or more medical conditions to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic Carelink™ Network.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94, or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. 5 computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in storage device 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuitry of computing devices 100 may implement any of the techniques described herein to analyze data received from IMD 10, e.g., to determine whether the health status of a patient has changed e.g., based on whether prediction criterion are satisfied and/or false prediction criterion are satisfied.

Storage device 96 may include a computer-readable storage medium or computer-readable storage device. In some examples, storage device 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

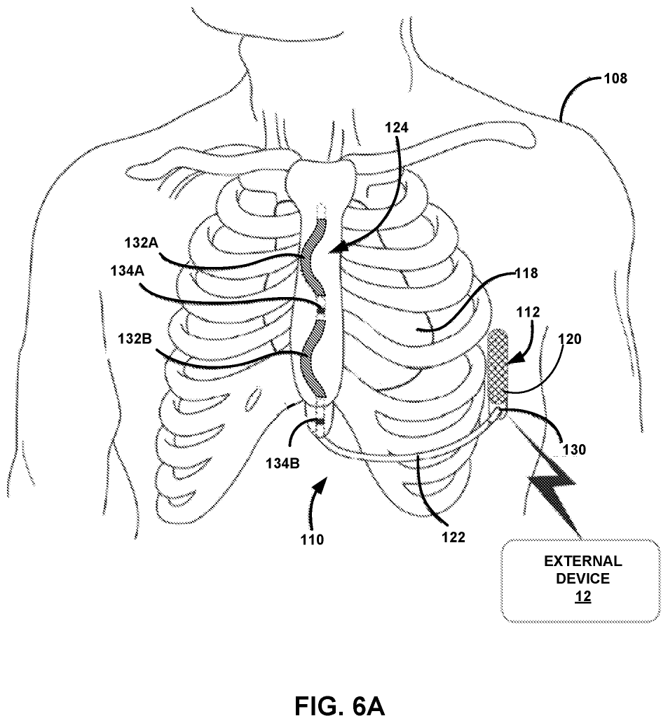

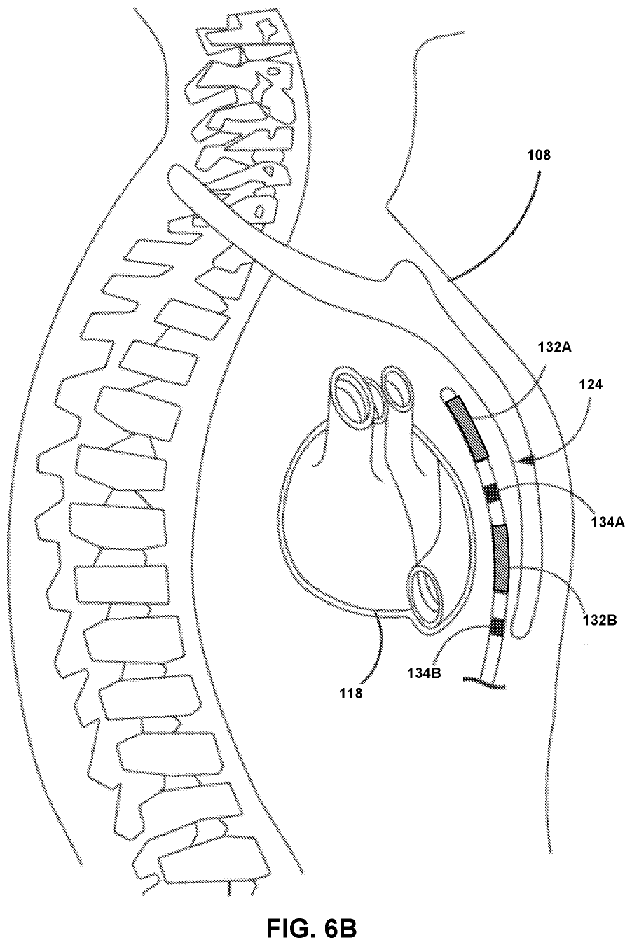

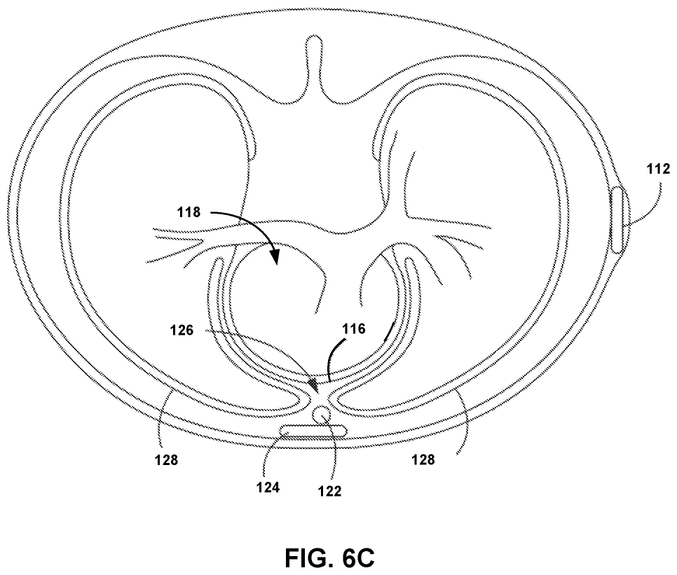

FIGS. 6A-6C are conceptual diagrams of another example medical system 110 implanted within a patient 108. FIG. 1A is a front view of medical system 110 implanted within patient 108. FIG. 1B is a side view of medical system 110 implanted within patient 108. FIG. 1C is a transverse view of medical device system 110 implanted within patient 108.

In some examples, the medical system 110 is an extravascular implantable cardioverter-defibrillator (EV-ICD) system implanted within patient 108. Medical system 110 includes IMD 112, which in the illustrated example is implanted subcutaneously or submuscularly on the left midaxillary of patient 108, such that IMD 112 may be positioned on the left side of patient 108 above the ribcage. In some other examples, IMD 112 may be implanted at other subcutaneous locations on patient 108 such as at a pectoral location or abdominal location. IMD 112 includes housing 120 that may form a hermetic seal that protects components of IMD 112. In some examples, housing 120 of IMD 112 may be formed of a conductive material, such as titanium, or of a combination of conductive and non-conductive materials, which may function as a housing electrode. IMD 112 may also include a connector assembly (also referred to as a connector block or header) that includes electrical feedthroughs through which electrical connections are made between lead 122 and electronic components included within the housing.

IMD 112 may provide the cardiac EGM sensing, asystole detection, and other functionality described herein with respect to IMD 10, and housing 120 may house circuitries 50-58, one or more sensor(s) 62, and an antenna 26 (FIGS. 2 and 3) that provide such functionality. Housing 120 may also house therapy delivery circuitry configured to generate therapeutic electric signals, such as cardiac pacing and anti-tachyarrhythmia shocks, for delivery to patient 108. System 110 may include an external device 12 that may function with IMD 112 as described herein with respect to IMD 10 and system 2.

In the illustrated example, IMD 112 is connected to at least one implantable cardiac lead 122. Lead 122 includes an elongated lead body having a proximal end that includes a connector (not shown) configured to be connected to IMD 112 and a distal portion that includes electrodes 132A, 132B, 134A, and 134B. Lead 122 extends subcutaneously above the ribcage from IMD 112 toward a center of the torso of patient 108. At a location near the center of the torso, lead 122 bends or turns and extends intrathoracically superior under/below sternum 124. Lead 122 thus may be implanted at least partially in a substernal space, such as at a target site between the ribcage or sternum 124 and heart 118. In one such configuration, a proximal portion of lead 122 may be configured to extend subcutaneously from IMD 12 toward sternum 24 and a distal portion of lead 22 may be configured to extend superior under or below sternum 124 in the anterior mediastinum 126 (FIG. 1C).

For example, lead 122 may extend intrathoracically superior under/below sternum 124 within anterior mediastinum 126. Anterior mediastinum 126 may be viewed as being bounded posteriorly by pericardium 116, laterally by pleurae 128, and anteriorly by sternum 124. In some examples, the anterior wall of anterior mediastinum 126 may also be formed by the transversus thoracis and one or more costal cartilages. Anterior mediastinum 126 includes a quantity of loose connective tissue (such as areolar tissue), some lymph vessels, lymph glands, substernal musculature (e.g., transverse thoracic muscle), and small vessels or vessel branches. In one example, the distal portion of lead 122 may be implanted substantially within the loose connective tissue and/or substernal musculature of anterior mediastinum 126. In such examples, the distal portion of lead 122 may be physically isolated from pericardium 116 of heart 118. A lead implanted substantially within anterior mediastinum 126 is an example of a substernal lead or, more generally, an extravascular lead.

The distal portion of lead 122 is described herein as being implanted substantially within anterior mediastinum 126. Thus, some of distal portion of lead 122 may extend out of anterior mediastinum 126 (e.g., a proximal end of the distal portion), although much of the distal portion may be positioned within anterior mediastinum 126. In other embodiments, the distal portion of lead 122 may be implanted intrathoracically in other non-vascular, extra-pericardial locations, including the gap, tissue, or other anatomical features around the perimeter of and adjacent to, but not attached to, the pericardium 116 or other portion of heart 118 and not above sternum 124 or the ribcage. Lead 122 may be implanted anywhere within the “substernal space” defined by the undersurface between the sternum and/or ribcage and the body cavity but not including pericardium 116 or other portions of heart 118. The substernal space may alternatively be referred to by the terms “retrosternal space” or “mediastinum” or “infrasternal” as is known to those skilled in the art and includes the anterior mediastinum 126. The substernal space may also include the anatomical region described in Baudoin, Y. P., et al., entitled “The superior epigastric artery does not pass through Larrey's space (trigonum sternocostale).” Surg.Radiol.Anat. 25.3-4 (2003): 259-62 as Larrey's space. In other words, the distal portion of lead 122 may be implanted in the region around the outer surface of heart 118, but not attached to heart 118. For example, the distal portion of lead 122 may be physically isolated from pericardium 116.

Lead 122 may include an insulative lead body having a proximal end that includes connector 130 configured to be connected to IMD 112 and a distal portion that includes one or more electrodes. As shown in FIG. 6A, the one or more electrodes of lead 122 may include electrodes 132A, 132B, 134A, and 134B, although in other examples, lead 122 may include more or fewer electrodes. Lead 122 also includes one or more conductors that form an electrically conductive path within the lead body and interconnect the electrical connector and respective ones of the electrodes.

Electrodes 132A, 132B may be defibrillation electrodes (individually or collectively “defibrillation electrode(s) 132”). Although electrodes 132 may be referred to herein as “defibrillation electrodes 132,” electrodes 132 may be configured to deliver other types of anti-tachyarrhythmia shocks, such as cardioversion shocks. Though defibrillation electrodes 132 are depicted in FIGS. 6A-6C as coil electrodes for purposes of clarity, it is to be understood that defibrillation electrodes 132 may be of other configurations in other examples. Defibrillation electrodes 132 may be located on the distal portion of lead 122, where the distal portion of lead 122 is the portion of lead 122 that is configured to be implanted extravascularly below the sternum 124.

Lead 122 may be implanted at a target site below or along sternum 124 such that a therapy vector is substantially across a ventricle of heart 118. In some examples, a therapy vector (e.g., a shock vector for delivery of anti-tachyarrhythmia shock) may be between defibrillation electrodes 132 and a housing electrode formed by or on IMD 112. The therapy vector may, in one example, be viewed as a line that extends from a point on defibrillation electrodes 132 (e.g., a center of one of the defibrillation electrodes 132) to a point on a housing electrode of IMD 112. As such, it may be advantageous to increase an amount of area across which defibrillation electrodes 132 (and therein the distal portion of lead 122) extends across heart 118. Accordingly, lead 122 may be configured to define a curving distal portion as depicted in FIG. 6A. In some examples, the curving distal portion of lead 22 may help improve the efficacy and/or efficiency of pacing, sensing, and/or defibrillation to heart 118 by IMD 112.

Electrodes 134A, 134B may be pace/sense electrodes (individually or collectively, “pace/sense electrode(s) 134”) located on the distal portion of lead 122. Electrodes 434 are referred to herein as pace/sense electrodes as they generally are configured for use in delivery of pacing pulses and/or sensing of cardiac electrical signals. In some instances, electrodes 134 may provide only pacing functionality, only sensing functionality, or both pacing functionality and sensing functionality. In the example illustrated in FIG. 6A and FIG. 6B, pace/sense electrodes 134 are separated from one another by defibrillation electrode 132B. In other examples, however, pace/sense electrodes 134 may be both distal of defibrillation electrode 132B or both proximal of defibrillation electrode 132B. In examples in which lead 122 includes more or fewer electrodes 132, 134, such electrodes may be positioned at other locations on lead 122.

In the example of FIG. 6A, the distal portion of lead 122 is a serpentine shape that includes two “C” shaped curves, which together may resemble the Greek letter epsilon, “ε.” Defibrillation electrodes 132 are each carried by one of the two respective C-shaped portions of the lead body distal portion. The two C-shaped curves extend or curve in the same direction away from a central axis of the lead body. In some examples, pace/sense electrodes 134 may be approximately aligned with the central axis of the straight, proximal portion of lead 122. In such examples, mid-points of defibrillation electrodes 132 are laterally offset from pace/sense electrodes 134. Other examples of extra-cardiovascular leads including one or more defibrillation electrodes and one or more pace/sense electrodes 134 carried by curving, serpentine, undulating or zig-zagging distal portion of lead 122 also may be implemented using the techniques described herein. In some examples, the distal portion of lead 122 may be straight (e.g., straight or nearly straight).

Deploying lead 122 such that electrodes 132, 134 are at the depicted peaks and valleys of serpentine shape may provide access to preferred sensing or therapy vectors. Orienting the serpentine shaped lead such that pace/sense electrodes 134 are closer to heart 118 may provide better electrical sensing of the cardiac signal and/or lower pacing capture thresholds than if pace/sense electrodes 134 were oriented further from heart 118. The serpentine or other shape of the distal portion of lead 122 may have increased fixation to patient 108 as a result of the shape providing resistance against adjacent tissue when an axial force is applied. Another advantage of a shaped distal portion is that electrodes 132, 134 may have access to greater surface area over a shorter length of heart 118 relative to a lead having a straighter distal portion.

In some examples, the elongated lead body of lead 122 may include one or more elongated electrical conductors (not illustrated) that extend within the lead body from the connector at the proximal lead end to electrodes 132, 134 located along the distal portion of lead 122. The one or more elongated electrical conductors contained within the lead body of lead 122 may engage with respective ones of electrodes 132, 134. The conductors may electrically couple to circuitry, such as a therapy delivery circuitry and sensing circuitry 52, of IMD 112 via connections in connector assembly. The electrical conductors transmit therapy from the therapy delivery circuitry to one or more of electrodes 132, 134, and transmit sensed cardiac EGMs from one or more of electrodes 132, 134 to sensing circuitry 52 within IMD 112.

In general, IMD 112 may sense cardiac EGMs, such as via one or more sensing vectors that include combinations of pace/sense electrodes 134 and/or a housing electrode of IMD 112. In some examples, IMD 112 may sense cardiac EGMs using a sensing vector that includes one or both of the defibrillation electrodes 132 and/or one of defibrillation electrodes 132 and one of pace/sense electrodes 134 or a housing electrode of IMD 112. Medical system 110, including processing circuitry of IMD 112 and/or external device 12, may perform any of the techniques described herein for determining whether prediction criterion (e.g., asystole detection criterion) are satisfied, e.g., based on physiological parameters sensed via sensor(s) 62. Medical system 110 may perform techniques that validate an initial detection (e.g., an initial asystole detection) as a true positive prediction or, in contrast, correct an initial detection to be a false positive prediction. Medical system 110 may validate or correct the initial detection e.g., based on cardiac EGMs sensed via extravascular electrodes 132, 134. As an alternative, medical system 110 may provide the initial detection based on cardiac EGMs sensed via extravascular electrodes 132, 134 and then, apply the prediction criterion to values for the patient's physiological parameters to either validate or correct that initial detection. In some examples, in response to an initial detection of a septic infection and based upon determining satisfaction of one or more false prediction criterion, medical system 110 may determine that the initial detection of a septic infection is a false positive. In other examples, medical system 110 may determine that the initial detection of a septic infection is a true positive based upon determining satisfaction of one or more true prediction criterion (or non-satisfaction with the one or more false prediction criterion).

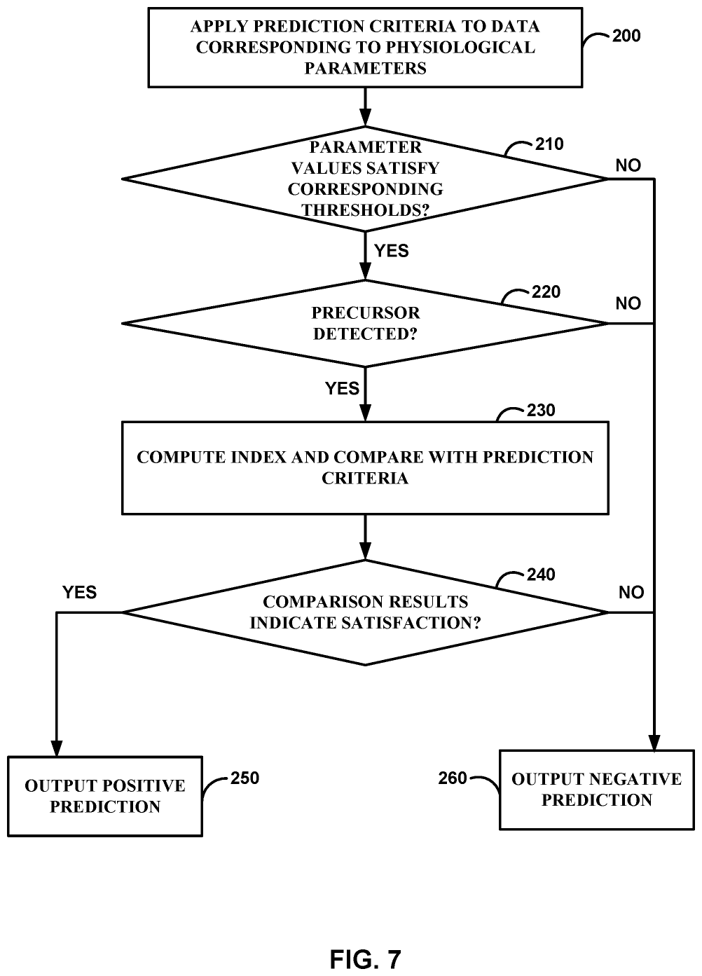

FIG. 7 is a flow diagram illustrating an example operation for determining changes in patient health. In some examples, the example operation may implement an algorithm for determining whether to render a prediction/detection based on whether a plurality of prediction criterion is satisfied (e.g., because a medical condition is afflicting the patient) and/or determining whether a prediction was false based on whether a plurality of false detection criteria is satisfied. According to the illustrated example of FIG. 7, processing circuitry of medical system 2, e.g., processing circuitry 50 of IMD 10, processing circuitry 80 of external device 12, and/or of one or more other computing devices, may apply at least one prediction criterion to data corresponding to physiological parameters sensed by one or more sensors 62 of IMD 10 (120). For example, as discussed in greater detail with respect to FIGS. 2-5, processing circuitry of medical system 2 may, in response to sensor data generated by sensing circuitry 52 of IMD 10, determine that a patient's physiological parameter data indicates a medical condition.

Sensing circuitry 52, via a plurality of electrodes of medical system 2 such as electrodes 132, 134 of FIGS. 6A-6C, may generate the sensor data from electrical signals produced by one or more sensors 62 while sensing the patient's physiological parameters. FIGS. 6A-6C depict examples where one or more leads facilitate the sensing of physiological parameters. FIG. 3 depicts leadless examples (e.g., pacemakers with housings configured for implantation within the patient's heart) where one or more sensors 62 do not rely upon leads to sense the patient's physiological parameters.

To detect medical conditions in view of the sensed patient physiological parameter data, processing circuitry of medical system 2 may build a data model defining each detectable medical condition, for example, in terms of one or more parameters (e.g., parameter values). Processing circuitry of medical system 2 may combine, into any example data model, any number of the patient's physiological parameters. To define a predictable medical condition to monitor for and possibly detect, processing circuitry of medical system 2 may use a single parameter, multiple parameters, a single parameter combining multiple parameters, and/or the like. Processing circuitry of medical system 2 may be configured to combine, into the example data models, the data corresponding to the plurality of physiological parameters with data corresponding to at least one of peripheral biological measurements or psychological assessments. Processing circuitry of medical system 2 may combine, into the example data model, peripheral biological measurements that comprise at least one of a weight or a pulse oximetry. Processing circuitry of medical system 2 may combine, into the example data model, psychological assessments such as quality of life or cognitive functions.

Once sufficiently built, processing circuitry of medical system 2 may apply a prediction algorithm (e.g., a sepsis prediction algorithm) to the example data model to generate, for instance, a sepsis index or score for comparison with various sepsis prediction criteria. It should be noted that medical system 2 may be hardcoded with any given sepsis prediction criterion and that criterion may be pre-determined and either immutable or mutable. Medical system 2 may receive the sepsis prediction criterion, via a network connection, from a remote monitoring service and that criterion may be static or dynamic. One criterion may be configured for application to a population or a sample group while another criterion may be narrowly tailored for the patient (e.g., the patient's physiology). The patient or another medical system 2 user may be permitted to adjust the criterion in some examples.

In the illustrated example, processing circuitry of medical system 2 determines whether one or more patient physiological parameter values satisfy corresponding threshold(s) (210). An example parameter value may refer to a patient physiological parameter such as those described herein; hence, the present disclosure may use a parameter value and a value determined for a physiological parameter interchangeably. Based on determining that any corresponding threshold is not satisfied (NO of 210), processing circuitry of medical system 2 proceeds to output a negative prediction (260).

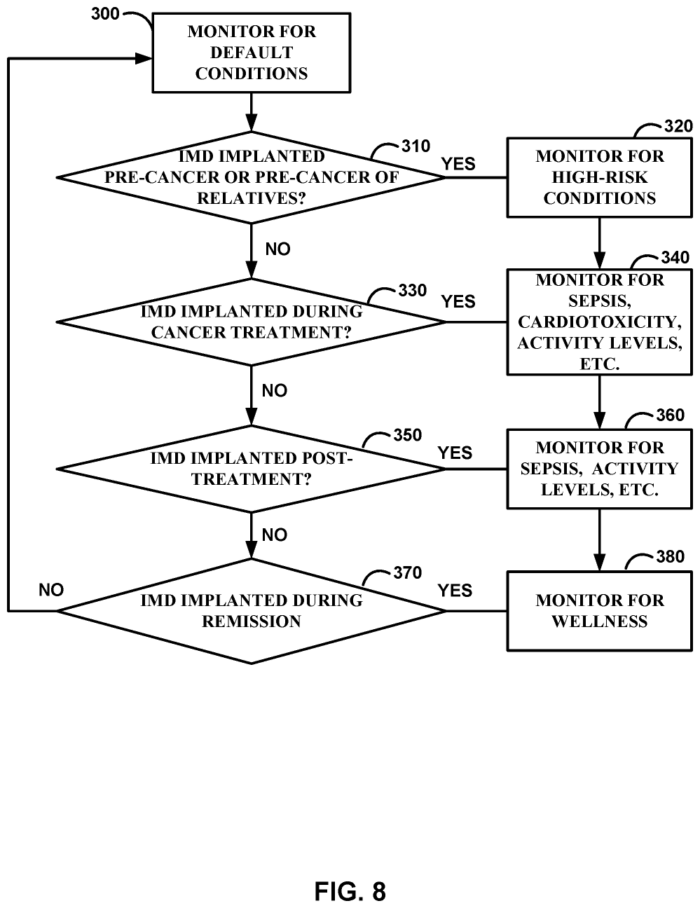

Based on determining satisfaction of the corresponding threshold(s) (YES of 210), processing circuitry of medical system 2 determines whether a precursor to a particular medical condition is detected (220). Detecting a precursor to sepsis, SIRS, may encompass fluid accumulation between one or more physiological parameters, such as temperature, heart rate, respiration rate, and activity steps or counts. Processing circuitry 80 of external device 12 may compare 2-6 parameters to individual parameter thresholds or incorporate the 2-6 parameters into an integrated algorithm to compute a score or index of SIRS detection. In some instances, only 2 of the 6 parameters must be elevated to transition a patient from SIRS to sepsis. Sepsis may be detected if the following one or more prediction criterion are met for at least X out of Y hours in a 24 hour period (e.g. X=3, y=12): Body Temperature >than 38° C. (100.4° F.) OR<36° C. (96.8° F.), AND Mean Heart Rate >90 bpm AND Respiratory rate >20 breaths per minute. Some examples of processing circuitry of medical system 2 may incorporate into the sepsis prediction criteria fluid accumulation and/or other parameters derived from impedance measurements and, as described in FIG. 8, may achieve suitable sensitivity and/or specificity. An integrated bioimpedance sensor in IMD 10 may advantageously provide values for fluid accumulation and other reliable parameters for detecting sepsis; eliminating any need to modify hardware/software in IMD 10, for example, to include additional sensors. Based on determining that the precursor is not detected (NO of 210), processing circuitry of medical system 2 proceeds to output a negative prediction (260).

Based on determining that the precursor is detected (YES of 220), processing circuitry of medical system 2 computes an index and compares the index with the one or more prediction criterion (230). In some examples, processing circuitry of medical system 2 computes the index as a mathematical combination of at least two parameters values (e.g., two or more values for a same parameter or values for two or more parameters). After computing the index, processing circuitry of medical system 2 determines whether comparison results indicate satisfaction of the prediction criterion (240). In response to determining satisfaction of the prediction criterion (YES of 240), processing circuitry of medical system 2 proceeds to output a positive prediction for the particular medical condition (250). Based on determining that at least one prediction criterion is not satisfied (NO of 240), processing circuitry of medical system 2 proceeds to output a negative prediction for the particular medical condition (260). In addition or as an alternative, if the computed index fails to satisfy the prediction criterion, processing circuitry of medical system 2 may generate for display output indicating the negative sepsis prediction for the patient or withhold the generated output until a determination that the prediction criterion is satisfied by an updated index. After either output, the example operation of FIG. 7 may end.

For example, processing circuitry 50 may determine whether the prediction criterion was satisfied at least a threshold number of times within a predetermined time period extending back from the most recent satisfaction of the prediction criterion, e.g., at least two times within the past thirty days. As another example, processing circuitry may determine whether the prediction criterion was satisfied at a threshold rate, e.g., a rate of one prediction per thirty days, over a time period. The time period may be the entire time IMD 10 has been active since implant, or since a period start time other than implant, e.g., a period starting a fixed number of days, weeks, or months after implant, or upon a power on reset or other reset of IMD 10.

As an optional step in lieu of ending the example operation of FIG. 7, based on determining that the at least one prediction criterion is satisfied, processing circuitry of medical system 2 proceeds to communicate one or more messages. These messages may include notifications of the positive prediction for receipt by a person or an entity with authority over the patient's therapy. Processing circuitry of medical system 2 may communicate (electronically) a message to email inboxes or directly to devices in use by the person or the entity or by the patient his/herself. The message may further include a control directive for the person or the entity to deliver the patient's therapy. Processing circuitry of medical system 2 may communicate a message to a device in control over the patient's therapy; and depending on the device, the message may include the notification of the positive sepsis prediction as well as an instruction for the device to deliver a dose of a treatment in accordance with a type and an amount.

As another optional step, based on determining that the one or more prediction criterion is satisfied (YES of 240), processing circuitry of medical system 2 proceeds to determine whether one or more false prediction criterion is not satisfied before outputting the positive prediction (250). Based on determining that the one or more false prediction criterion is satisfied, the example operation of FIG. 7 may end after processing circuitry of medical system 2 withholds the positive prediction and instead, outputs the negative prediction (260).

Based on the example operation of FIG. 7 ending, e.g., due to none of the false prediction criterion being satisfied, or an insufficient number or combination of the false prediction criterion being satisfied, processing circuitry 50 may classify the suspected medical condition as a true prediction. Based on the prediction being classified as true, processing circuitry 50 may use the medical condition prediction in further operations, such as calculating statistics, or transmitting true prediction data to other devices. Based on determining that the prediction of the suspected medical condition is a false prediction, processing circuitry 50 may use the false prediction in further operations, such as calculating statistics of false predictions and transmitting false prediction data to other devices, e.g., for consideration by a user of a modification of the operation of IMD 10 to avoid further false predictions.

The order and flow of the operation illustrated in FIG. 7 is one example. In other examples according to this disclosure, more or fewer prediction criterion may be considered, the prediction criterion may be considered in a different order, or satisfaction of different numbers or combinations of prediction criterion may be required for a prediction of the particular medical condition and/or a determination that the prediction of the particular medical condition (e.g., the suspected septic infection) was false. Further, in some examples, processing circuitry may perform or not perform the method of FIG. 7, or any of the techniques described herein, as directed by a user, e.g., via external device 12 or computing devices 100. For example, a patient, clinician, or other user may turn on or off functionality for identifying false asystole detection remotely (e.g., using Wi-Fi or cellular services) or locally (e.g., using an application provided on a patient's cellular phone or using a medical device programmer).

Additionally, although described in the context of an example in which IMD 10, and processing circuitry 50 of IMD 10, perform each of the portions of the example operation, the example operation of FIG. 7, as well as the example operations described herein with respect to FIG. 7, may be performed by any processing circuitry of any one or more devices of a medical system, e.g., any combination of one or more of processing circuitry 50 of IMD 10, processing circuitry 80 of external device 12, processing circuitry 98 of server 94, or processing circuitry of computing devices 100. In some examples, processing circuitry 50 of IMD 10 may sample data corresponding to the patient's physiological parameters for determining whether the prediction criterion is satisfied, and provide that sampled data for evaluation with the prediction criterion to another device. In such examples, processing circuitry of the other device, e.g., external device 12, server 94, or a computing device 100, may apply one or more prediction criterion to the sampled data.

FIG. 8 is a flow diagram illustrating an example operation for monitoring a patient's physiological parameters for medical conditions. The example operation, described herein with reference to medical system 2 and discussed in greater detail with respect to FIGS. 1-5, may, in response to (samples of) sensor data generated by sensing circuitry 52 of IMD 10 and for each medical condition, apply one or more prediction criterion and determine whether the patient has a sufficient likelihood of having that medical condition. In some examples, the example operation may implement an algorithm for determining whether to render a prediction/detection based on whether the one or more prediction criterion are satisfied (e.g., because a medical condition is afflicting the patient) and/or determining whether a prediction was false based on whether a plurality of false detection criteria is satisfied. The example operation is operative to adjust a criterion and/or apply a different prediction criterion depending on the patient's current health status.

In some examples, the patient (e.g., a cancer patient) and their health may experience a series of temporal stages (e.g., over a time period encompassing the patient's cancer diagnosis and recovery) and at each stage, the example operation may invoke different prediction criteria, for example, for monitoring specific medical conditions. The example operation of FIG. 8 may commence at any one of these stages and as the cancer patient progresses through treatment, IMD 10 may monitor the patient's physiological parameters for susceptible infections and diseases and some may be more prevalent at one stage than another stage.

According to the illustrated example of FIG. 8, processing circuitry of medical system 2, e.g., processing circuitry 50 of IMD 10, processing circuitry 80 of external device 12, and/or of one or more other computing devices, may apply at least one prediction criterion to data corresponding to one or more of the patient's physiological parameters for each default medical condition to monitor (300). In some examples, upon successfully implanting IMD 10 into the patient, the processing circuitry configures IMD 10 with default settings for detecting cardiac issues (e.g., an arrhythmia) and other maladies. In case the processing circuitry cannot identify the patient's current health status, the processing circuitry invokes the default settings for operating IMD 10. In some examples, the processing circuitry may apply prediction/detection criteria for routine diseases and infections, such as bacterial, viral, and other parasitic infections (e.g., influenza) and any disease caused by such infections. In these examples, the processing circuitry may identify these routine diseases and infections with ample time for medicinal intervention because curbing the severity of any infection/disease at the pre-cancer stage may alleviate at least some pain and suffering when the cancer symptoms appear or prevent the pain and suffering from worsening. Additionally, early detection of such infections could avoid repeat surgeries and device change outs, resulting in improved quality of care for the patient, prevention of worsening of illness, and decreased cost to the healthcare system.

The following description of the example operation is in reference to an implantation of IMD 10 into a cancer patient. The processing circuitry may determine whether IMD 10 is being/has been implanted into the cancer patient before that patient or one of that patient's relatives has cancer (i.e., pre-cancer stage) (310). At the pre-cancer stage, the patient may be onset with early signs/symptoms of cancer, or the patient is cancer-free. The patient's genetic profile may or may not indicate a genetic proclivity for cancer. Based on determining that the patient is at the pre-cancer stage (YES Branch of 310), the processing circuitry may proceed to monitor the patient for high-risk conditions (320), for example, because the patient may be (currently) healthy and therefore, capable to manage low-risk conditions. The example operation may transition from a current stage to a next stage based on the patient's health status; for instance, in accordance with the example operation, when that current stage ends, the processing circuitry may halt monitoring the patient for the current stage's medical conditions and proceed to commence monitoring for the next stage's medical conditions. In some examples, the processing circuitry may determine that there has been a recent administration of treatment on the patient and in turn, end the monitoring for high-risk conditions at the pre-cancer stage and start monitoring the patient for medical conditions that are specific to the cancer stage; and when the cancer stage ends, the processing circuitry may start monitoring the patient for medical conditions that are specific to the post-cancer stage and so forth. IMD 10 may receive user input (e.g., from the patient) and/or data input (e.g., database records such as electronic medical records (EMR) and in response, determine an appropriate stage and activating the monitoring of medical conditions corresponding to that stage.

Based on determining that the patient is not at the pre-cancer stage (NO Branch of 310), the processing circuitry may proceed to determine whether IMD 10 is being/has been implanted into the patient during/before cancer treatment (i.e., cancer stage) (330). Based on determining that the patient is at the cancer stage and currently receiving treatment such as chemotherapy (YES Branch of 330), the processing circuitry may proceed to monitor the patient for sepsis, cardiotoxicity, activity levels, and/or other medical conditions/parameters (340).

A large percentage of cancer patients are affected by a septic infection due to their immunocompromised state, and early prediction could allow for prevention and improve mortality of cancer patients. As described herein and for FIG. 6, the processing circuitry may predict that the patient most likely has a septic infection by first detecting SIRS and/or ARDS, the infection's precursor, based on satisfaction of one or more prediction criterion for septic infections. For each SIRS prediction criterion, the processing circuitry may determine satisfaction, for example, by evaluating a condition for, detecting a presence of, or comparing a threshold to one or more physiological parameters including parameters corresponding to temperature, fluid accumulation and other parameters based on sensed impedance (e.g., in IMD 10), heart rate, respiration rate, patient activity, and/or the like. In some examples, the processing circuitry executes an integrated algorithm incorporating any combination of these parameters integrated algorithm to compute a score predicting SIRS for the patient. In one example, the processing circuitry may output data indicating a positive septic infection prediction if the integrated algorithm detects an elevation in three or more of the above parameters and that elevation is non-trivial/statistically significant and indicative of SIRS.

Similarly, the processing circuitry may apply one or more prediction criterion to determine whether the patient most likely has cardiotoxicity. For example, for medical system 2 to accurately predict cardiotoxicity, the processing circuitry (e.g., in IMD 10) may measure surrogate of left ventricular volume, e.g., distance between leads on a 2-3 lead device. Cardiotoxicity may be defined when there is a decline in one or more dimensions measured, which would be a surrogate for the cardiotoxicity metric: A decline in Left ventricular ejection fraction (LVEF) of at least 5% to less than 55% with signs or symptoms of Congestive heart failure (CHF), a decline in LVEF of at least 10% to below 55% without signs or symptoms, AT interval prolongation, an increase in Premature ventricular contractions (PVC) or arrhythmia (e.g., non-sustained Ventricular Tachycardia (VT)) burden, changes in QRST morphologies (particularly for repolarization related features), and/or the like. In some examples, the processing circuitry may apply various prediction criteria for detecting other infections (e.g., viral infections such as influenza).

IMD 10 is configured to monitor (in real-time) the patient's cardiac activity and based on certain sensor data (e.g., a cardiac electrogram (EGM) depicting suspected episode data)), detect certain cardiac events (e.g., arrhythmia). The processing circuitry may be configured to leverage current detection/monitoring logic in IMD 10 to better titrate and manage chemotherapy treatment (e.g., for personalized titration of the chemotherapy agent(s)). The processing circuitry may compute a titration score using a multi-variate algorithm taking into account not only cardiac-related parameters but other parameters indicating different aspects of patient's health current status. Some example parameters are indicative of a worsening prognosis for the patient, such as a decrease in activity or change in gait pattern, symptoms, or significant variability in weight gain. The processing circuitry may communicate directly to a therapy delivery/drug dispensing mechanism for pharmacologic treatments. This mechanism may be an internal or an external device.

In accordance with the example operation, the processing circuitry may apply IMD 10's native functionality to benefit pediatric patients undergoing chemotherapy. The processing circuitry may employ IMD 10 to engage in vital sign monitoring and data collecting (e.g., automatically on an hourly basis) where alerts may be communicated to user devices (e.g., operated by parents or doctors) and/or network devices (e.g., operated by a remote monitoring service) if, for example, a threshold is crossed. To highlight an example of such an alert, if a parameter (e.g., temperature) exceeds a preset threshold (e.g., 103° F.), IMD 10 communicates an alert directly to the patient's medical provider. Other vitals to be measured include (1) Heart rate, (2) Temperature, (3) Respiration, (4) Activity, (5) Fluid Status/accumulation. A variety of inputs may provide physiological information corresponding to a particular vital sign. The Activity vital may body position (e.g., orientation or pose) and/or body movement. In addition or as an alternative to alerting the parent or the medical provider, the processing circuitry may communicate with a drug dispensing device to automate drug dissemination to alleviate cancer symptoms.

Based on determining that the patient is not at the cancer stage (NO Branch of 330), the processing circuitry may proceed to determine whether IMD 10 is being/has been implanted at/by completion of the patient's cancer treatment (i.e., post-cancer stage) (350). Based on determining that the patient is at the post-cancer stage (YES Branch of 350), the processing circuitry may proceed to monitor the patient for sepsis, activity levels, and/or other medical conditions/parameters (360). Based on determining that the patient is not at the post-cancer stage (NO Branch of 350), the processing circuitry may proceed to determine whether IMD 10 is being/has been implanted into the patient during remission (i.e., cancer remission stage) (370).

Based on determining that the patient is at the cancer remission stage (YES Branch of 370), the processing circuitry may proceed to monitor the patient for wellness (380). Wellness monitoring during remission, in general, encompasses a broad range of maladies, infections, diseases, and other medical conditions that if left untreated, could complicate the patient's cancer recovery. Wellness monitoring may include routine health issues, such as early onset heart failure and cardiovascular issues. In some example, the processing circuitry may turn on IMD 10 periodically (monthly or semi-annually) to assess the patient's overall heart health including (1) electrocardiogram (ECG) recording, (2) heart rate, (3) arrhythmia assessment, and (4) any surrogate value of echo measurement, such as filling parameters (E and A waves) or measure of left ventricular wall thickness derived from impedance measurements and remotely transmits to the following physician.

IMD 10 may monitor the patient's heart for rhythm abnormalities, fluid abnormalities and/or the like. Assessment of fluid status (e.g., from impedance sensing), arrhythmia monitoring, including QRS width and p-wave sensing, and other device diagnostics to attribute a heart failure risk score to cancer patients who are in remission on a monthly basis. The processing circuitry may turn on IMD 10 every 30 days for the first 5 years and produce an overall heart health score. Following the first 5 years, the processing circuitry may transition the example operation to annual monitoring. If an arrhythmia or other variable was detected which was out of range, IMD 10 would remain on and recording until a physician had reviewed the data to make a diagnosis.

Wellness monitoring may extend to lymphatic system. In one example, by monitoring the patient's fluid status, the processing circuitry may determine whether the lymphatic system was unable to properly remove fluid in an affected region of the body. Inability to remove fluid would result in potential cellulitis, edema, or other complications. If IMD 10 is implanted in locations of the patient where lymph nodes have been removed as a result of medical intervention, e.g., removal of cancerous lymph nodes. The processing circuitry may configure IMD 10 with thresholds corresponding to the accumulation of fluids. Lymphatic system monitoring may be configured in a leadless IMD 10, and would include thresholds to detect lymph fluids. This system may also utilize the capability to measure pressure to assess edema severity as part of the alerting capability. In addition, temperature and pressure measurements would be used to assess the probability of infection. Incapacity of the lymphatic system can lead to infection. Sepsis is also associated with fluid accumulation and may be detected with good sensitivity from an integrated bioimpedance sensor in IMD 10.

Based on determining that the patient is not at the cancer remission stage (NO Branch of 370), the processing circuitry may determine that a current stage cannot be identified and/or execute default logic operative to apply one or more prediction criterion for each of one or more default medical conditions (300). IMD 10 may be pre-configured with each prediction criterion (e.g., default setting). In some examples, the processing circuitry may halt device setup of IMD 10 and/or generate, for display, content indicative of a device error in IMD 10.

As an option, the example operation may adjust, add, or remove criterion from use in monitoring a specific medical condition including any of the above-described medical conditions. A number of medical conditions utilize or rely (at least in part) on fluid-related parameters including fluid status/accumulation. The processing circuitry of medical system 2 may be predict likely instances (e.g., infections) of sepsis and other medical conditions with sufficient sensitivity/specificity from an integrated bioimpedance sensor in IMD 10. To accomplish such sensitivity/specificity, processing circuitry of medical system 2 may be configured to improve upon the prediction criteria's accuracy. As described herein, amongst a plurality of physiological parameters, employing fluid accumulation in the prediction criteria may result in sufficient sensitivity/specificity.

Although sensitivity and specificity of any detector are important, perhaps the most relevant index of accuracy for this application is the positive predictive value (PPV=TP/TP+FP), or the percentage of predictions that are correct. PPV is influenced by the prevalence of disease in the population that is being tested. If used in a high prevalence setting, it is more likely that persons who test positive truly have disease than if the test is performed in a population with low prevalence. Since the accuracy of the detection algorithm will depend on the population, tight specification of the test population will be vital to algorithm performance and can compensate somewhat for relatively low specificity.

Sensitivity has never really been an issue for impedance and parameters corresponding to impedance measurements. Indeed, high sensitivity has hampered adoption as impedance parameters can detect a myriad of morbidities including heart failure, anemia, salt overload, weight loss/gain, drug non-adherence etc. For specificity, one obvious method to make improvements would be to add additional orthogonal sensors such as temperature and heart rate as described in the disclosure.

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an 1 MB, an external programmer, a combination of an 1 MB and external programmer, an integrated circuit (IC) or a set of ICs, and/or discrete electrical circuitry, residing in an IMD and/or external programmer.

What is claimed is:

1. A medical system comprising:
one or more sensors configured to sense a plurality of physiological parameters for a patient;
sensing circuitry coupled to the one or more sensors and configured to generate sensor data comprising data indicative of the plurality of physiological parameters comprising an impedance parameter corresponding to fluid accumulation; and
processing circuitry configured to:
compute an infection index based upon values corresponding to the impedance parameter and at least one other of the plurality of physiological parameters; and
based upon a comparison between the infection index and infection prediction criterion, generate, for display, output data corresponding to the comparison results, wherein the output data indicates a prediction of infection in the patient if the comparison results indicate satisfaction of the infection prediction criterion.

2. The medical system of claim 1, wherein the one or more sensors are configured to sense signals corresponding to the plurality of physiological parameters, wherein the signals comprise information associated with impedance, cardiac electrogram, acceleration, temperature, or optical coherence.

3. The medical system of claim 1, wherein each parameter of the physiological parameters corresponds to at least one of a patient activity, a body temperature, a respiration rate, a tidal volume index, a heart rate, a heart rate variability, an arrhythmia burden, a fluid accumulation index, a blood pressure, or a glucose level,
wherein, to determine the infection index, the processing circuitry is configured to determine whether a respective level of each physiological parameter satisfies a corresponding criterion.

4. The medical system of claim 1, wherein the processing circuitry is further configured to:
based upon a determination that the infection index satisfies the infection prediction criterion, generate for display output indicating a positive infection prediction for the patient, or
based upon a determination that the infection index fails to satisfy the infection prediction criterion, generate for display output indicating a negative infection prediction for the patient or withhold the generated output until a determination that the infection prediction criterion is satisfied by an updated infection index.

5. The medical system of claim 1, wherein the processing circuitry is further configured to:
compute at least one of a difference between the infection index and a second infection index or a rate of change infection indices over time; and
based upon a determination that at least one of the computed difference or the computed rate of change exceeds an infection threshold, generate for display the output indicating the infection prediction for the patient.

6. The medical system of claim 1, wherein the processing circuitry is configured to:
combine, into a data model, the data corresponding to the plurality of physiological parameters with data corresponding to at least one of peripheral biological measurements or psychological assessments; and
apply a infection prediction algorithm to the data model to generate the infection index.

7. The medical system of claim 6, wherein at least one of the peripheral biological measurements comprise at least one of weight or pulse oximetry or the psychological assessments comprise quality of life or cognitive functions.

8. The medical system of claim 1, wherein the one or more sensors are further configured to sense at least one of the physiological parameters of a patient via a plurality of electrodes of the medical system.

9. The medical system of claim 1, wherein the processing circuitry is further configured to communicate at least one of a message to a person or an entity with authority over the patient's therapy or a message to a device in control over the patient's therapy, the message comprising at least one of a notification of a positive infection prediction, a control directive to apply the patient's therapy, an instruction to deliver a dose of a treatment wherein the instruction specifies a type and an amount of the dose.

10. The medical system of claim 1, wherein the processing circuitry is further configured to execute a monitoring algorithm for the patient, wherein the monitoring algorithm, in accordance with a schedule, causes the medical system to:
capture signals corresponding to one or more of the plurality of physiological parameters;
update the infection index based upon data representative of the captured signals; and
compare the infection index with the infection prediction criterion.

11. A method, comprising:
processing sensor data comprising data indicative of a plurality of physiological parameters for a patient comprising an impedance parameter;
computing an infection index based upon values corresponding to the impedance parameter and at least another one of a plurality of physiological parameters; and
based upon a comparison between the infection index and infection prediction criterion, generating, for display, output data corresponding to the comparison results, wherein the output data comprises data indicative of a prediction of infection in the patient if the comparison results indicate satisfaction of the infection prediction criterion.

12. The method of claim 11, further comprising:
in response to the comparison results, performing at least one of
based upon a determination that the infection index satisfies the infection prediction criterion, generating for display data indicating a positive infection prediction for the patient, or
based upon a determination that the infection index fails to satisfy the infection prediction criterion, performing at least one of generating for display data indicating a negative infection prediction for the patient or withholding the generated output until a determination that the infection prediction criterion is satisfied by an updated infection index.

13. The method of claim 11, further comprising:
computing at least one of a difference between the infection index and a second infection index or a rate of change infection indices over time; and
based upon a determination that at least one of the computed difference or the computed rate of change exceeds an infection threshold, generating for display the output indicating the infection prediction for the patient.

14. The method of claim 11, further comprising:
combining, into a data model, the data corresponding to the plurality of physiological parameters with data corresponding to at least one of peripheral biological measurements or psychological assessments; and
applying an infection prediction algorithm to the data model to generate the infection index.

15. The method of claim 11, further comprising:
communicating a message to a device with control over the patient's therapy, the message causing the device to administer a treatment.

16. The method of claim 11, wherein each parameter of the physiological parameters corresponds to at least one of a patient activity, a body temperature, a respiration rate, a tidal volume index, a heart rate, a heart rate variability, an arrhythmia burden, a fluid accumulation index, a blood pressure, or a glucose level,
wherein determining the infection index further comprises determining whether a respective level of each physiological parameter satisfies a corresponding criterion.

17. The method of claim 11, further comprising:
computing a titration score using a multi-variate algorithm based on a dataset comprising at least one of the plurality of physiological parameters and the comparison results based upon the comparison between the infection index and the infection prediction criterion; and
performing at least one of adjusting a current treatment or determining a new treatment for the patient based on the titration score.

18. The method of claim 17, wherein the dataset is indicative of a worsening prognosis or improving prognosis for the patient.

19. The method of claim 17, wherein the dataset comprises one or more physiological parameters corresponding to a decrease in patient activity, a change in gait pattern, a change in one or more symptoms, or a substantial weight gain or weight loss.

20. A non-transitory computer-readable storage medium comprising program instructions that, when executed by processing circuitry of a medical system, cause the processing circuitry to:
process sensor data comprising data indicative of a plurality of physiological parameters for a patient comprising an impedance parameter;
compute a sepsis index based upon values corresponding to an impedance parameter and at least one other of the plurality of physiological parameters; and
based upon a comparison between the sepsis index and sepsis prediction criterion, generate, for display, output data corresponding to the comparison results, wherein the output data indicates a prediction of sepsis in the patient if the comparison results indicate satisfaction of the sepsis prediction criterion.
